(19) United States
(12) Patent Application Publication
(10) Pub. No.: US 2006/0275800 A1
(43) Pub. Date: Dec. 7, 2006

(54) Process for characterization of the transactivation and transrepression activity of glucocorticoid receptor ligands in primary immune cells

(76) Inventors: Wolf Dietrich Doecke, Berlin (DE); Christine Rentzsch, Berlin (DE); Heike Schaecke, Berlin (DE); Norbert Schmees, Berlin (DE); Hartmut Rehwinkel, Berlin (DE); Bettina Schulz, Berlin (DE); Patrick Golletz, Berlin (DE); Christine Stock, Jena (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/414,550

(22) Filed: May 1, 2006

Related U.S. Application Data
(60) Provisional application No. 60/679,993, filed on May 12, 2005.

(30) Foreign Application Priority Data
Apr. 29, 2005 (DE) 10 2005 020 711.1

(51) Int. Cl. G06F 19/00; C12Q 1/68; G01N 33/53

(52) U.S. Cl. 435/6; 435/7.1; 702/19; 702/20

(57) ABSTRACT

The invention relates to a process for the characterization of the transactivation and transrepression activity of glucocorticoid receptor (GR) ligands by gene and/or protein expression analysis in primary immune cells as well as the use thereof.

The invention relates to a process for characterization of the transactivation and transrepression activity of glucocorticoid receptor (GR) ligands by gene and/or protein expression analysis in primary immune cells.

Glucocorticoids are included in the medications used most frequently in clinical practice. They have significant actions on the inflammatory and immune system and on the metabolism. The various actions are mediated by different mechanisms via the glucocorticoid receptor (GR). Selective glucocorticoid receptor (GR) ligands represent a new class of GR ligands, which selectively agonistically or antagonistically influence the GR mechanisms of gene regulation, the transactivation and the transrepression of genes. A dissociation of the transactivation activity and the transrepression activity of GR ligands or of agonistic and antagonistic actions of GR ligands is possible. In this respect, the therapeutic profile of the GR ligands is to be selectively influenced.

For in vitro characterization of the GR ligands with respect to the activity in the transactivation and in the transrepression, human and animal cell lines are currently used that are transfected in some cases with promoter constructs. Selective individual aspects of the GR actions are queried in these cell lines. Moreover, a stimulation of the cells is often necessary for the detection of the corresponding activity of the GR ligands. A conclusion from the results of such assays in cell lines on the actions of the GR ligands in primary cells and in particular on the human system is possible only to a very limited extent.

For in vivo characterization of GR ligands with respect to their molecular mechanism, on the one hand, functional tests are used. Examples in this respect are the application of GR ligands when an inflammation is triggered or the detection of actions of the GR ligand in the wake of a metabolic stress or after administration of the metabolism-regulating hormones. In studies on humans, this approach is associated with major problems or is not feasible. On the other hand, the GR-ligand-mediated gene regulation is examined directly in the extracted target organs or in organ samples under experimental conditions. This approach is generally not possible for studies on humans.

Based on the above-mentioned, significant limitations of the current assays, the development of assays that 1) have a greater relevance for the characterization of GR-ligand actions in primary cells and 2) can be used for the characterization of the molecular mechanism of GR ligands in studies on humans, without producing a significant stress, is desired.

The idea was to define genes in primary immune cells that reflect the transactivation and transrepression activity of GR ligands in their regulation.

To this end, genes were selected in a screening with selective GR ligands with defined transactivation and transrepression activity in unstimulated human primary immune cells of the peripheral blood, which reflect the transactivation or transrepression activity of the substances in a quick, reproducible and consistent manner in the suppression or in the induction of the gene expression. The regulation of the selected genes by the selective GR ligands was further characterized in the dose/action and kinetic studies. Kinetic studies in the animal model confirm the reproducibility of the gene regulation under in vivo conditions.

The suppression of the IL-1β, IL-8, Rantes and in particular TNF-α gene expression has proven especially suitable for the detection of the transrepression activity of GR ligands in unstimulated primary immune cells. The induction of the expression of glutamine synthetase and GILZ, and quite especially that of CD163 and FKBP51, has proven especially suitable for the detection of the transactivation activity.

With the parameters, it was possible to characterize both agonistic and antagonistic effects, both in vitro and also after in vivo application, of selective GR ligands or standard glucocorticoids on the transactivation or transrepression activity of the GR. The use of these parameters for characterizing the molecular mechanism of GR ligands is novel.

The following advantages can be expected from the use of the gene and/or protein expression analysis in unstimulated primary immune cells for detecting the molecular mechanism of GR ligands:
1) Primary immune cells are a non-artificial cell system with great relevance for the actions of the GR ligands in the organism.
2) The detection of parameters in unstimulated primary immune cells avoids the necessity of stimulations that could falsify the parameters and not reflect the situation in the organism.
3) The studies confirm that the parameters in the primary immune cells reflect the molecular mechanism of GR ligands in a quick, dose-dependent, reproducible and consistent manner both after in vitro addition and after in vivo administration.
4) The very good agreement between the in vitro results obtained on unstimulated primary human immune cells and the results obtained in animal-trial studies after in vivo administration of GR ligands makes the applicability of the defined parameters for in vivo studies on humans very likely.
5) The detectability of parameters in unstimulated primary immune cells also allows a direct determination of parameters in blood samples of organisms that are treated with GR ligands. Thus, the study material is readily accessible for in vivo studies.
6) The parameters can be determined in blood samples of organisms that are treated with GR ligands without causing additional stress in taking blood. An additional intervention for administering the corresponding GR ligands and for blood sampling is not necessary. The absolutely required blood volume for the studies is less than one milliliter.
7) The parameters can be detected in whole-blood assays so that an impairment of the results, e.g., by a cell separation, takes place. In this respect, commercially available detection systems are available.
8) The detection of the parameters in unstimulated primary immune cells can be used both for experimental in vitro and in vivo studies and for Phase I and clinical studies. In this respect, a consistency of the parameters for the molecular mechanism of GR ligands is possible in the discovery, the development and in the clinical use of GR ligands.
9) The parameters are suitable as biomarkers for the characterization of the molecular in vivo mechanism of GR ligands in studies on humans.
10) The selected parameters for the transrepression (suppression of the expression of the IL-1β, IL-8, Rantes and in particular TNF-α inflammation mediators) allow in addition a characterization of the anti-inflammatory and immunomodulatory action of the GR ligands.
11) Since the transactivation and transrepression activity decisively determines the actions of GR ligands in the organism, the characterization of the defined gene and/or protein expression parameters in the unstimulated primary immune cells of the peripheral blood can help to obtain, even in early studies and even on healthy humans (e.g., Phase I studies), information on the molecular in vivo mechanism and thus the action/side effect profile of the GR ligand that is to be expected.

Primary immune cells in terms of the patent are all immune cells of the living organism. In particular, the immune cells of the blood, the bone marrow and the lymphatic organs (e.g., thymus, spleen, lymph nodes, Peyer's plaque) are meant. The immune cells of the blood are quite especially preferred.

Principle of the Method

The method describes the characterization of the molecular mechanism of glucocorticoid receptor (GR) ligands (standard glucocorticoids and selective GR ligands) by means of analysis of the gene and protein expression of GR-sensitive genes in primary immune cells.

Selective GR ligands represent a new class of GR ligands, which employ the two mechanisms of the GR-mediated regulation of the gene expression, the transactivation or the transrepression of sensitive genes, to a varying extent. These can be agonists, partial agonists, partial antagonists and antagonists for the respective mechanism. Corresponding to the GR-mediated anti-inflammatory, immunosuppressive and metabolic actions, indications of selective GR ligands can be, i.a., inflammatory diseases or conditions with an altered metabolic activity. An essential requirement for the discovery, the successful development and the clinical use of such new GR ligands is the characterization of its molecular mechanism in relevant in vitro and in vivo experiments and in Phase I and clinical studies on humans.

The purpose of the method, in experimental in vitro or in vivo studies as well as in Phase I and clinical studies on humans, is to characterize the transrepression and transactivation activity of selective GR ligands and standard glucocorticoids by changes of the gene and/or protein expression or protein release in unstimulated immune cells, in lymphatic organs and in the blood. To this end, parameters were selected in a screening from over 50 genes and were analyzed in more detail. Essential selection criteria were 1) the correlation of the gene regulation with the transrepression or transactivation activity of selective GR ligands, 2) the consistent gene regulation in unstimulated primary immune cells, and 3) the quick reaction in the in vitro assays as well as the good and enduring detectability after in vivo administration of GR ligands. These criteria are essential for the use of the parameters as biomarkers.

Both for in vitro detection and for ex vivo detection, the suppression of the pro-inflammatory IL-1β, IL-8, Rantes and in particular TNF-α cytokines has proven especially suitable for the characterization of the transrepression activity, and the induction of glutamine synthetase and GILZ and quite especially that of CD163 and FKBP51 have proven especially suitable for the transactivation activity.

Other suitable parameters are the expression of co-accessory molecules (HLA-DR, CD86) for the detection of the transrepression and the expression of cytokine receptors (IFN-γR1, TNF-R1, IL-1R1, IL-2Rα, IL-13Ra, CXCR4, GITR) as well as other genes (β2-adrenoreceptor, hemoxygenase 1, IL-2, MIF, annexin 1, thrombospondin 1) for the detection of the transactivation activity.

The detection of the regulation of the expression of the cited parameters can be carried out by methods for the mRNA detection (e.g., quantitative real-time PCR) and/or for the protein detection (e.g., continuous-flow cytometry, immunoassays, Western Blot) in the primary immune cells. Secreted proteins can be detected in culture supernatants, serum, plasma and other biological fluids. The detection methods are known to one skilled in the art in this field—they are, moreover, adequately described in the technical literature so that their implementation is possible.

The applicability of the method is documented below based on the characterization of the standard glucocorticoidal prednisolone as well as two selective GR agonists (SEGRA) of the Schering AG. The differential mechanism of the substances in the usual assays for the agonistic or antagonistic transactivation and transrepression activity in the GR correlates with the regulation of the expression of selected genes in in vitro assays of primary human immune cells and in the spleen cells of substance-treated mice.

The assays for the GR-ligand-mediated regulation of the gene and/or protein expression can be used both for detecting competitive or non-competitive agonistic and antagonistic activity of substances for the transactivation or transrepression mechanism of the GR. For the latter, the antagonizing of the actions of another added GR ligand is studied. In in vivo experiments, in addition the antagonizing of the endogenic glucocorticoid actions can be characterized.

The method that is shown is suitable for the characterization of the molecular mechanism of GR ligands in in vitro and in in vivo experiments and in biomarker assays for Phase I and clinical studies on humans.

1 Regulation and Action of Endogenic Glucocorticoids

1.1 Regulation of Endogenic Glucocorticoids

The adrenal glands of the healthy adult produce between 40 and 80 μmol (15 and 30 mg; 8-10 mg/m²) of endogenic cortisone daily. The plasma concentration is determined by the secretion, the inactivating rate and the formation of free cortisol and shows a clear circadian profile. The circadian cycle, interactions with the autonomous nervous system and with physical and emotional stress, as well as the reaction with hypoglycemia and systemic inflammation are controlled via the regulation of the adrenal cortisone production by the hypothalamic-pituitary-suprarenal cortex (HPA)-axis. The hypothalamic “corticotropin-releasing hormone” (CRH) that is triggered by the above-mentioned stimuli induces the production of the “adrenocorticotropic hormone” (ACTH) from the pituitary gland, which increases the cortisone synthesis by stimulation of the suprarenal cortex. Within the framework of a negative feedback regulation, the systemically increased gluocorticoid levels inhibit the CRH synthesis and the ACTH release.

The biological effects of endogenic and iatrogenic glucocorticoids in the body depend not only on the cortisone-plasma levels. In addition, they are regulated by the two isoforms of an enzyme system, the 11β-hydroxy steroid dehydrogenases (11β-HSD). The 11β-HSD I catalyzes the conversion of biologically inactive cortisone in active cortisol. Conversely, the 11-β-HSD II supports the conversion of active cortisol in inactive cortisone. The system is primarily located in the liver but also in other tissues, such as in fatty tissue (Tomlinson, J. W.; Endocr. Rev. 2004, 25:831).

1.2 Mechanism of the Action of Glucocorticoids by the GR

The actions of glucocorticoids are mediated/transferred via the glucocorticoid receptor (GR). The GR belongs to the protein family of the core receptors, which are activated after the binding of their respective ligands as transcription factors and have influence on the expression of specific target genes. The binding of the ligand to the GR that is present in the cytoplasma of the cell induces a change in the receptor conformation, which in turn has the result of a translocation of the now ligand-bonded GRs in the cell nucleus. There, the activated GR is able to have a positive or negative influence on the expression of the target genes.

In the case of a positive regulation of the gene expression (transactivation), the GR is bound as a homodimer to specific sequences (glucocorticoid response elements; GREs) in the promoter of sensitive genes. The fact that the dimerization of the GR is a requirement for the transactivation was indicated by mutation analyses in vitro and in vivo (Reichardt, H. M., Cell. 1998, 93:531; Heck, S., EMBO J. 1994, 13:4087). According to more recent knowledge, however, it must be stated, restrictively, that many, but not all GR-induced transactivation processes are dependent on the dimerization of the receptor (Rogatsky I, PNAS 2003, 100:13845).

The ligand-activated GR is also able to inhibit the expression of specific target genes (transrepression). The most common mechanism of the negative regulation is carried out by binding the activated GR as a monomer to other transcription factors that are already bonded to the DNA. By this binding, the activity of the other transcription factors is suppressed and thus also the expression of the target gene. In another mechanism of the negative regulation of the gene expression, the binding of the activated GR to the so-called negative GREs, which can be found in promoters of several genes, is carried out. In this connection, by the binding of the GR, there results a displacement of other transcription factors that are essential for the induction of the expression of the gene. Thus, the binding of the GR to the nGREs prevents the transcription of the corresponding target genes.

In addition, the GR is able to tackle, in an inhibiting manner, the MAP kinases in specific signal transfer paths and in this way to mediate its effects.

1.3 Important GK Actions and Mechanisms

Metabolic GK Actions

Many physiological processes, such as the regulation of the glucose balance, protein and fat metabolism, are controlled by glucocorticoids. In many of these physiological processes, glucocorticoids act by exerting influence on the expression of involved proteins/enzymes (Wang, M., Nutr. Metab. (Lond). 2005, 2:3). The liver-specific phosphenol pyruvate-carboxykinase (PEPCK) and also the glucose-6-phosphatase are expressed to an increased extent by glucocorticoids. The expression of the glucose transporter GLUT4 is also induced. (Imai, E., Mol. Cell. Biol. 1990, 10:4712; Schmoll, D., FEBS Lett. 1996, 383:63; Lin, B., DNA Cell Biol. 1998, 17:967; Grosfeld, A., Diabetologia 2002, 45:527).

An increased amino acid catabolism, which reliably represents the energy supply of the organism in hunger situations, results from the expression, enhanced by glucocorticoids, of, e.g., the tyrosinaminotransferase (TAT) (Becker et al., 1986; Jantzen et al., 1987), the glutamine synthetase or the tryptophanoxygenase (Becker, P. B., Nature 1986, 324; 686; Schmid, E., Eur. J. Biochem. 1987, 165:499; Danesch, U., EMBO J. 1987, 6:625; Gaunitz, F., Biochem. Biophys. Res. Commun. 2002, 296:1026).

The intervention in the fat balance by glucocorticoids is also carried out by the regulation of the expression of some proteins/enzymes that are involved in the fat balance. It thus was shown that the hormone-sensitive lipase HSL and the lipoprotein lipase LPL are highly regulated by glucocorticoids (Zilberfarb, V., Diabetologia. 2001, 44:377). The same applies for the proteins leptine and VLDLR (“very low density lipoprotein receptor”) that are involved in the energy or lipometabolism (Slieker, L. J., J. Biol. Chem. 1996, 271, 5301; Ensler, K., Biochim. Biophys. Acta. 2002, 1581:36).

Anti-Inflammatory and Immunosuppressive Actions

Glucocorticoids achieve anti-inflammatory and immunomodulatory activity by engaging in inflammatory signal transfer paths. This happens either by inhibiting the activity of cells of the adaptive or innate immune system or by direct disruption of the pro-inflammatory, cytokine-controlled signal transfer paths. The transrepression has been described as a main mechanism for the anti-inflammatory and immunosuppressive activity of glucocorticoids. Via the inhibition of the activity of several transcription factors (in particular of NF-κB and AP-1), the preparation of many pro-inflammatory cytokines (e.g., TNF-α, GMCSF, IL-1β, IL-2, IL-3, IL-12), chemokines (e.g., IL-8, RANTES, eotaxin, MIP), enzymes (iNOS, COX-2) and/or adhesion molecules (ICAM-1, VCAM-1) is reduced (Barnes, P. J., Clin. Sci. (Lond). 1998, 94:557, Almawi, W. Y., J. Mol. Endocrinol. 2002, 28:69).

Also, however, the induction of anti-inflammatory proteins (lipocortin-1, serum leucoprotease inhibitor, neural endopeptidase, MKP-1) is involved in the anti-inflammatory action of glucocorticoids (Barnes, P. J., Clin. Sci. (Lond.), 1998, 94:557; Kassel, O., EMBO J. 2001, 20:7108).

An involvement in the anti-inflammatory and immunosuppressive actions of glucocorticoids is also attributed to other, non-genomic effects, such as the interruption of MAP-kinase-signal paths.

1.4 Side Effects of Glucocorticoids

The above-mentioned physiological and anti-inflammatory and/or immunosuppressive actions of glucocorticoids can lead to the chronic excess of existing glucocorticoids (e.g., in the case of endogenic hypercortisolism or therapeutic glucocorticoid administration) but also to a number of undesirable effects, such as the induction of hyperglycemia to triggering diabetes mellitus, high blood pressure, muscular atrophy and/or myopathy, truncal obesity, osteoporosis, i.a.

Special effects that are involved in the glucose and fat balances are regulated in a significant portion by glucocorticoid-induced transactivating processes.

In addition to the above-mentioned enzymes, other enzymes that are involved in the protein catabolism, such as, e.g., the glutamate dehydrogenase, the glutamate-oxalazetate-transaminase and the serine-dehydratase, are induced by glucocorticoid administration (Timmerman, M., Exp. Biol. Med. (Maywood) 2003, 228:100; Barouki, R., Eur. J. Biochem. 1989, 186:79; Su, Y., Arch Biochem. Biophys. 1992, 297:239). A permanent highly regulated gluconeogenesis can result in hyperglycemia, insulin resistance and, as a consequence, diabetes mellitus. As mentioned above, the key enzymes of the gluconeogenesis in the liver are induced by glucocorticoids.

A constant immunosuppression by chronic glucocorticoid treatment or an endogenic hypercortisolism can lead to an increased risk of infection. The mechanisms that are involved in the above are essentially those that are made responsible for the therapeutic actions (anti-inflammatory and/or immunosuppressive). In addition to the inhibition of the expression of many pro-inflammatory proteins, however, the GR is also involved in an increased risk of infection by a direct induction of viral promoters.

2 Selective GR Ligands and Indications for their Use

2.1 Selective GR Ligands

Selective GR ligands (e.g., selective GR agonists—SEGRA, Selective GR Modulators—SGRM, dissociated or differentiated GR ligands) represent a new class of GR ligands that operate the two mechanisms for regulation of gene expression, the transactivation or the transrepression of sensitive genes, to a varying extent (Schaecke, H., PNAS 2004, 101:227 & Curr. Opin. Investig. Drugs. 2004, 5:524). In this connection, in comparison to the endogenic glucocorticoids and/or therapeutic standard glucocorticoids, comparable, increased, reduced and eliminated actions in the transactivation or in the transrepression and their different combinations are possible. These can be agonists, partial agonists, partial antagonists and antagonists for the respective mechanism. While the agonistic actions are defined by the direct effects of the GR ligands on the expression of sensitive genes and/or promoter constructs, the antagonistic actions are characterized via the inhibition of the effects of other ligands on GR. Corresponding sample assays in this respect are presented below in the characterization of the molecular mechanism of two selective GR agonists (SEGRA) from the WO 00/32584 compound 1 and from the WO 02/10143 compound 2 of the Schering AG.

For the transactivation, agonistic ligands of the GR activate the receptor by their binding in the form so that the latter is able to induce their transcription by binding to the GREs in the promoter regions of sensitive genes. In addition, there are ligands that cause an antagonistic conformation of the receptor by binding to the GR and do not result in an induction of the promoter activities of sensitive genes. Such ligands are referred to as antagonists. Another group of ligands can be referred to as partial agonists or partial antagonists. The latter induce only partially agonistic or antagonistic GR activities. The antagonistic or partial-agonistic activities of GR ligands are based on the cellular background and the structure of the promoter, which regulates the expression of sensitive genes.

For the transrepression, agonistic ligands of the GR activate the receptor by their bond in the form so that the ligand-activated receptor is able to inhibit the expression of specific target genes. This can be carried out by interaction and subsequent inhibition of the action of other transcription factors or by binding to negative GREs. Partial agonists, partial antagonists and antagonists in the transrepression mediate this GR action only partially and/or inhibit the agonistic action of other GR ligands in the transrepression.

2.2 Sample Indications for the Treatment with Selective GR Ligands

A treatment with selective GR ligands has its indications, on the one hand, wherever standard glucocorticoids are indicated. Here, a dissociated action on the GR can lead to an advantage of the selective GR ligands with respect to an induction of undesirable actions that are reduced in comparison to standard glucocorticoids, i.e., to an improved action/side effect profile. On the other hand, selective GR ligands can be used to antagonize the adverse effects of existing GR ligands. In addition, a selective substitution of inadequate actions of the endogenic glucocorticoids by selective GR ligands is possible, without the side effects of undesirable effects at the GR occurring.

These treatment principles can also occur in combination. Sample applications are given below.

Anti-Inflammatory and Immunosuppressive Treatment

Glucocorticoids belong to the most commonly used anti-inflammatory and immunosuppressive medications (Franchimont, D., Ann NY Acad Sci. 2004, 1024:124). Their use is limited by, however, in some cases serious and non-reversible side effects. It has been shown that the transrepression activity of the GR is essential for the anti-inflammatory and immunosuppressive action of GR ligands, while important side effects (e.g., induction of gluconeogenesis) are mediated by the transactivation (Schaecke, H., Pharmacol. Ther. 2002, 96:23).

Selective GR ligands with a reduced transactivation activity in the transrepression activity that is obtained could produce effective anti-inflammatory and immunosuppressive medications that induce side effects that are reduced in comparison to standard glucocorticoids and that essentially are mediated via the transactivation mechanism.

Treatment of the Endogenic Hypercortisolism

A pathophysiological significance of a, in some cases, tissue-specific, increased activity of the endogenic glucocorticoids is discussed for a number of diseases and syndromes (e.g., diabetes mellitus, truncal obesity, metabolic syndrome, hypertonia, arteriosclerosis). These syndromes are characterized by increased metabolic actions of the endogenic glucocorticoids (i.a., hyperglycemia, hyperlipoidemia) with the adverse results thereof. Moreover, a chronic inflammation (low-level inflammation) is often found (Li, J. J., Medical Hypotheses 2005, 64:236; Wang, M., Nutr. Metab. (Lond). 2005, 2:3, Dandona, P., Circulation, 2005, 111: 1448). A treatment with the now approved standard glucocorticoids is not indicated, however, based on the increase of the undesirable metabolic effects that are to be expected.

Selective GR ligands with a (partial)-antagonistic action in the transactivation could reduce the adverse metabolic actions of the endogenic glucocorticoids via different mechanisms. These include the inhibition of the endogenic glucocorticoid synthesis via the inhibition of the hypothalamic-pituitary-suprarenal cortex axis and the competitive and/or non-competitive antagonizing of the metabolic actions of the endogenic glucocorticoids in the GR. Moreover, an agonism of the selective GR ligands in the transrepression could control the chronic inflammation.

Treatment of Conditions with a Deficiency of Metabolic Actions

In various predominantly serious diseases, the additional metabolic actions of glucocorticoids are desired. These include cachexia, e.g., in tumors, cardiovascular diseases or HIV infection. These conditions are also characterized by an immunodepression, however, which would be enhanced by a treatment with standard glucocorticoids (Mulligan, K., Int J Cardiol. 2002, 85, 151; Tijerina, A. J., Dimens Crit Care Nurs. 2004, 23:237).

Selective GR ligands with a pronounced agonistic action in transactivation and a (partial) antagonism in the transrepression could induce the desired metabolic actions of the GR, without impairing the defense situation of the organism. Even an improved infection defense that results by the antagonizing of the transrepression actions of the endogenic glucocorticoids would be conceivable.

3 Characterization of the Molecular Mechanism of Selective GR Ligands in Primary Immune Cells

3.1 Schematic Representation

The examples above show that selective GR ligands with different transactivation/transrepression profiles could be used in a number of indications. A significant requirement for the discovery, the successful development and for the clinical use of such new GR ligands is the characterization of their molecular mechanism in relevant in vitro and in vivo experiments and in Phase I and clinical studies on humans. The nucleated cells of the peripheral blood, i.e., the peripheral blood leukocytes, and the proteins derived therefrom, are especially suitable for the monitoring of the transactivation- and transrepression-mediated gene regulation by selective GR ligands and standard glucocorticoids. Significant advantages compared to other assays for the characterization of the molecular mechanism of GR ligands are 1) the use of unstimulated primary cells, which have relevance for the in vivo action of glucocorticoids, as well as 2) the simple applicability as biomarkers in studies on humans (detection in the blood, without invasive interventions, e.g., biopsies, or other additional stresses).

The direct detection of the gene regulation in the primary immune cells can be carried out by the mRNA detection, e.g., by an RT-PCR or other amplification methods or by methods for direct mRNA detection. For the detection of protein, e.g., the continuous-flow cytometry, immunoassays or Western Blot methods can be used. The detection of proteins can be performed on or in the cells, in the cell lysates or in the supernatants of cell cultures or in plasma, serum or other biological liquids.

The purpose of the methodology is to characterize the molecular mechanism of GR ligands based on the regulation of the expression of genes and/or proteins in primary immune cells and thus to obtain indications on the possible action profile of the substances that are used.

Regarding the definition of suitable parameters, the regulation, mediated by standard glucocorticoids and selective GR ligands, of more than 50 genes in primary human immune cells was examined in a broad screening approach. Selected genes were further characterized in vitro and in vivo in kinetic experiments. The conditions deviating from the literary works resulted in some cases in inverse results. Decisive criteria for the parameter selection were 1) the consistent reflection of transactivation or transrepression properties of GR ligands, 2) the detection of the regulation in non-stimulated immune cells, and 3) the quick reaction of the gene expression in in vitro assays (the 4-hour time period was used for the screening), and the good and enduring detectability after in vivo application of GR ligands. These properties make it likely that a direct regulation of the gene expression by the GR ligands will take place and that the parameters will be usable in in vivo studies (e.g., as biomarkers).

To have a comparison with a standard GK, the GR-ligand-influenced gene expressions for these parameters were normalized to the gene expression after treatment with the standard GK, prednisolone. Prednisolone is the standard GK, in comparison to which the SEGRA substances are to exhibit an increased dissociation and which could represent the comparison substance in the case of clinical studies with SEGRA development candidates. It is clear to one skilled in the art that the normalization could also in principle be related to any other glucocorticoid.

For gene suppression, the “prednisolone/GR ligand” quotient was derived for purposes of normalization, i.e., a lower value (<1) indicates a relatively weaker suppression by the GR ligands (higher values in the residual gene expression). For gene induction, the “Gr-ligand/prednisolone” quotient was derived for purposes of normalization, i.e., a value <1 shows a relatively weaker induction by the GR ligands.

### In Vitro mRNA Expression of Selected Transactivation and Transrepression Parameters Normalized to the Prednisolone Treatment

| | Transactivation vs. | | Transrepression | |
|---|---|---|---|---|
| | Prednisolone | | vs. Prednisolone | |
| | CD163 | FKBP51 | IL-1β | TNF-α |
| Prednisolone 1 Hour | 1.00 | 1.00 | 1.00 | 1.00 |
| Prednisolone 2 Hours | 1.00 | 1.00 | 1.00 | 1.00 |
| Prednisolone 4 Hours | 1.00 | 1.00 | 1.00 | 1.00 |
| Prednisolone 12 Hours | 1.00 | 1.00 | 1.00 | 1.00 |
| Prednisolone 24 Hours | 1.00 | 1.00 | 1.00 | 1.00 |
| Dexamethasone 1 Hour | 1.02 | 0.89 | 0.61 | 0.80 |
| Dexamethasone 2 Hours | 1.89 | 1.13 | 0.69 | 0.85 |
| Dexamethasone 4 Hours | 1.34 | 1.08 | 1.27 | 1.01 |
| Dexamethasone 12 Hours | 1.13 | 1.07 | 0.96 | 1.00 |
| Dexamethasone 24 Hours | 1.04 | 1.05 | 1.16 | 0.97 |
| ZK 238587 1Hour | 0.84 | 0.99 | 1.16 | 0.99 |
| ZK 238587 2 Hours | 0.52 | 0.93 | 0.87 | 0.86 |
| ZK 238587 4 Hours | 0.49 | 0.79 | 1.28 | 0.89 |
| ZK 238587 12 Hours | 0.48 | 0.74 | 0.97 | 1.06 |
| ZK 238587 24 Hours | 0.49 | 0.82 | 0.73 | 0.79 |
| ZK 243149 1 Hour | 0.81 | 0.91 | 0.87 | 0.96 |
| ZK 243149 2 Hours | 0.42 | 0.68 | 0.73 | 0.72 |
| ZK 243149 4 Hours | 0.21 | 0.58 | 0.73 | 0.75 |
| ZK 243149 12 Hours | 0.39 | 0.63 | 0.87 | 0.76 |
| ZK 243149 24 Hours | 0.27 | 0.68 | 0.41 | 0.49 |
| ZK 243185 1 Hour | 0.93 | 0.77 | 1.17 | 0.76 |
| ZK 243185 2 Hours | 0.33 | 0.19 | 0.51 | 0.59 |
| ZK 243185 4 Hours | 0.04 | 0.26 | 1.25 | 0.73 |
| ZK 243185 12 Hours | 0.07 | 0.26 | 0.45 | 0.67 |
| ZK 243185 24 Hours | 0.06 | 0.33 | 0.85 | 0.52 |

The purpose was to characterize the extent of the dissociation of a substance by a value that reflects the varying influence of the expression of different transrepression and transactivation parameters in a summary.

To characterize the dissociated action of the SEGRA test substances, a dissociation factor was therefore defined that shows the dissociation of transactivation and transrepression activity based on the induction or suppression of selected genes.

Below, a ratio between transrepression and transactivation parameters from the prednisolone-normalized values was derived according to the following formula:

$$Ratio = \frac{\sqrt[n]{{\prod }_{t = 1}^{n} {TR}_{t}}}{\sqrt[m]{{\prod }_{r = 1}^{m} {TA}_{r}}} \qquad Formula 1$$

In this formula:
TR stands for the normalized transrepression parameter (TR_{prednisolone}=1)
TA stands for the normalized transactivation parameter (TA_{prednisolone}=1)

$$\sqrt[m]{M}$$

stands for the m-th root of M

Example: √{cube root}\sqrt{27}=3

$${\prod }_{x = 1}^{t} {T}_{x}$$

stands for the product of all values T_{X}, whereby the index x runs from 1 to t

Example:

$${\prod }_{x = 1}^{5} {T}_{x} = {T}_{1} \times {T}_{2} \times {T}_{3} \times {T}_{4} \times {T}_{5}$$

The ratio is thus the n-th root of the product of n different prednisolone-normalized transrepression parameters divided by the m-th root of the product of m different prednisolone-normalized transactivation parameters.

As transrepression parameters, all known parameters are suitable, including the expression of co-accessory molecules (HLA-DR, CD86). Especially suitable parameters are IL-1β, IL-8, Rantes—TNF-α is especially preferred.

As transactivation parameters, all known parameters are suitable, including the expression of cytokine receptors (IFN-γR1, TNF-R1, IL-1R1, IL-2Rα, IL-13Ra, CXCR4, GITR) as well as other genes (β2-adrenoreceptor, hemoxygenase 1, IL-2, MIF, annexin 1, thrombospondin 1). Especially suitable parameters are the expression of glutamine synthetase and GILZ and quite especially suitable are those of CD163 and FKBP51.

Any can be selected from these transrepression and transactivation parameters and can be used according to the above-mentioned formula for determining the ratio according to formula 1.

TNF-α and IL-1β are preferably selected for determining the transrepression. CD163 and FKBP51 are preferably selected for determining the transactivation.

According to this preferred combination of parameters, a ratio between transrepression parameters and transactivation parameters is derived after normalization of the individual parameters according to formula 2 below:

$$Ratio = \frac{\sqrt[2]{TNF - α \times IL - 1 β}}{\sqrt[2]{CD 163 \times FKBP 51}} \qquad Formula 2$$

### EXPERIMENTAL PART

The results in the gene or protein expression obtained in the experimental part below were obtained by means of quantitative real-time RT-PCR or by means of continuous-flow cytometry and are plotted as mean value±standard deviation.

For the mRNA detection, the total RNA was isolated from the respective samples (human PBMC or human whole blood or spleen cells from mice), transcribed in the cDNA and amplified and detected below by means of Real-time TaqMan-PCR (Applied Biosystems). A relative quantification in comparison to the expression of the “house-keeping” gene HPRT was carried out. In each case, the ratio of the result for the GR ligands to the result of the vehicle control is plotted.

The continuous-flow-cytometric detection of the expression of receptors in the human immune cells was performed with commercially available fluorescence-labeled monoclonal antibodies from the corresponding receptor proteins. The labeling was carried out in whole blood. The continuous-flow-cytometric analysis was carried out after the erythrocytes were lysed by means of a FACScalibur-continuous-flow cytometer (Becton Dickinson). The average fluorescence activity is plotted.

The tested compounds are compound 1 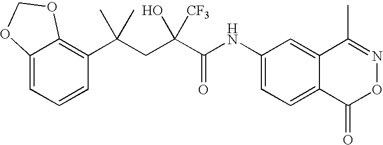
from WO 00/32584 and compound 2 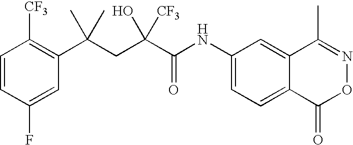
from WO 03/082827.
3.2 Gene/Protein Suppression as Parameters for the Transrepression Activity

Inhibition of the expression by standard glucocorticoids is known for many genes in the immune cells (Galon, J., FASEB, J. 2002, 16:61; Hayashi, R., Eur. J. Pharmacol. 2004, 500:51).

In our studies on unstimulated primary immune cells, genes/proteins that play a role in the inflammation and the specific immune response have proven suitable as indicators for the transrepression activity. These parameters, moreover, have the advantage that their inhibition reflects the anti-inflammatory and immunosuppressive effects of GR ligands. The figure below shows the gene regulation, detected by means of quantitative real-time-PCR, of inflammatory cytokines (TNF-α, IL-1β, Il-8, Rantes) and the co-stimulatory molecule CD86 by the standard glucocorticoid prednisolone in 4-hour cultures of unstimulated human peripheral mononuclear blood cells (PBMC).

[image]

The in vivo action of prednisolone on the expression of genes (TNF-α, IL-1β, Rantes), inhibited by standard glucocorticoids, was studied in the spleen cells of mice after 24 hours.

[image]

A sample parameter for the reduction of the spontaneous protein expression, which reflects the anti-inflammatory and immunosuppressive actions of GR ligands, is the MHC class 11 (HLA-DR) expression on the monocytes that is determined by continuous-flow cytometry in a whole blood assay.

[image]

The mRNA expression of IL-1β, IL-8 (only available in the human system), Rantes, and in particular that of TNF-α has proven especially suitable for the in vitro and ex vivo detection of the transrepression activity of GR ligands in primary, unstimulated immune cells.

3.3 Gene and/or Protein Induction as Parameters for the Transactivation Activity

An induction of genes by standard glucocorticoids in immune cells is known (Galon, J., FASEB, J. 2002, 16:61).

For the genes identified in our screening below, we could detect a correlation of the induction in unstimulated immune cells with the transactivation properties of selective GR ligands: cytokine receptors (e.g., TNF-R1, IFN-γR1, IL-1R1, IL-2Rα, IL-13Ra, GITR, CXCR4), CD163, FKBP51, annexin 1, IL-2, β2-adrenoreceptor, MIF, GILZ, hemoxygenase 1, thrombospondin 1 and glutamine synthetase. These proteins are involved, in some cases, essentially in the anti-inflammatory and metabolic actions of GR ligands and in the regulation of the GR actions. Results opposite to those of the literature were obtained in some cases, thus, e.g., a repression, not induction, of the expression of IL-1RA was observed (not shown).

The gene and/or protein expression of CD163, FKBP51, GILZ and the glutamine synthetase proved especially suitable for the in vitro and ex vivo detection of the transactivation activity of GR ligands.

The figure shows the induction of genes by prednisolone in 4-hour cultures of human PBMC.

[image]

The figure below of selected parameters for the transrepression activity and for the transactivation activity of GR ligands shows the dose-dependency of the effect of prednisolone on the gene expression.

[image]

The in vivo induction of the expression of selected genes was detected in the spleen cells of mice treated with prednisolone.

[image]

[Key: Glutamin synthetase=Glutamine Synthetase]

Below, the increasing effects of prednisolone on the protein expression of the receptors on the monocytes in human whole-blood cultures, detected by continuous-flow-cytometry, are plotted. Similar results were found for the granulocytes.

[image]

In summary, studies of both the gene expression and the protein expression in immune cells are suitable for characterizing the inhibiting (transrepression) or increasing effects (transactivation) of GR ligands. The correlation of such results with the results of transactivation and transrepression screening assays is shown subsequently.

4 Selective GR Agonists (SEGRA) from WO 00/32584 and WO 02/10143

Below, the assays for the characterization of the molecular mechanism of the SEGRA substances in the screening as well as the results for two selected SEGRA substances with different transactivating activity are plotted.

These assays document the status of now common processes for characterizing the molecular mechanism of GR ligands based on receptor assays or promoter assays in cell lines.

4.1 Characterization of the Molecular Mechanism of GR Ligands

First, the substances undergo receptor binding tests to show the binding to the GR and simultaneously the selectivity for the GR.

The binding of the substances to the glucocorticoid receptor (GR) and other steroid hormone receptors (mineralocorticoid receptor (MR), progesterone receptor (PR) and androgen receptor (AR)) is examined with the aid of recombinantly produced receptors. To this end, extracts from SF9 cells that were infected with baculoviruses that contain the coding sequences for the respective steroid hormone receptor are used. In comparison to the reference substance [³H]-dexamethasone, the substances show a high to very high affinity to the GR.

To determine the transactivating activities of SEGRA substances, two test systems are used.

The promoter of the mouse mammary tumor virus (MMTV) contains specific binding sites for the activated GR (so-called GREs). This promoter was cloned before a reporter gene (luciferase), and the construct was integrated in a stable manner in the genome of the human cell line HeLa (cervix carcinoma cells). By adding test and reference substances, the MMTV promoter is activated, and the luciferase, whose activity can be detected by means of photometric measurement, is expressed.

In a second transactivation system, the induction of the tyrosinaminotransferase (TAT) by glucocorticoids is determined. In the promoter of the gene for the TAT, GREs are also located, so that this gene is expressed in an enhanced form by binding of the ligand-activated GR. To this end, rat hepatoma cells (H4IIE3) are treated for 24 hours with test and reference substances, and then the TAT activity is determined by photometry. For the two assays, both the detection of agonistic actions of the SEGRA substances in the transactivation and the detection of antagonistic actions in the induction of the parameters by other GR ligands are possible.

To determine the activity in the transrepression, a promoter system that contains portions of the collagenase promoter is used. The promoter was placed before a reporter gene (luciferase), and the construct that was produced was integrated in stable form into the genome of the human cell line HeLa. After the cells are stimulated with phorbol ester, this promoter is activated. The administration of SEGRA test substances and glucocorticoids inhibits the phorbol ester-induced promoter activity. The detection is carried out via the photometric determination of the luciferase activity.

The activities of SEGRA substances in the respective transactivation and transrepression systems are determined in comparison to the activities of the reference substance dexamethasone.

4.2 Transactivation Agonist Compound 1

The SEGRA-substance compound 1 had been tested as an agonist in the two above-mentioned transaction assays. Compound 1 induces the activity of the MMTV promoter with a power of 10±1.4 nmol and an effectiveness of 73±2.8% of the maximum dexamethasone effect (n=2). In the induction of the TAT activity in the rat hepatoma cells, the substance shows a power of 5.7±0.6 nmol and an effectiveness of 86±12.7% of the maximum dexamethasone effect (n=2). It has an antagonistic effect neither in the MMTV promoter assay nor in the TAT promoter assay.

4.3 Transactivation-Antagonist Compound 2

In contrast to compound 1, compound 2 is a clear antagonist relative to the MMTV promoter. The substance antagonizes the MMTV promoter activity that is induced by dexamethasone at a power of 85±12 nmol and an effectiveness of 119±3.6% of the maximum effect of the GR antagonist RU 486 (n=3). In the TAT promoter, compound 2 behaves like a partial agonist. The activity of the TAT promoter is induced by compound 2 at a power of 67±10 nmol and an effectiveness of 43.5±10.6% (n=2) of the maximum dexamethasone effect. At a concentration of 1 μmol, compound 2 antagonizes the dexamethasone-induced TAT activity at 35% of the maximum effect of RU 486.

5 Characterization of the Transactivation and Transrepression Activity of Compound 1 and Compound 2 by Means of Gene Expression Analysis in Primary Immune Cells

Below, by way of example, the application of the characterization of the transactivation and transrepression activity of selective GR ligands by means of gene expression analysis in primary immune cells is shown comparatively based on two SEGRA substances, the agonist in the transactivation, compound 1, and the antagonist in the transactivation, compound 2. The molecular mechanism of these substances had been previously characterized in the usual assays (see 5.2 and 5.3). The gene expression analysis was performed by means of quantitative Real-Time-TaqMan-PCR. The ratios (mean value±standard deviation) of the results for the GR ligands to the results for the vehicle control are shown.

5.1 In Vitro Results in Primary Human Immune Cells

The SEGRA substances show a comparable inhibition of the expression of cytokines in human PBMC as parameters for the transrepression activity.

[image]

In contrast to this, the different transactivation profile of the SEGRA substances is reflected in differing degrees of gene induction. While the agonist, compound 1, clearly induces the genes, the antagonist in the transactivation, compound 2, results in lower gene induction or no gene induction.

[image]

[Key: Glutamin synthase=Glutamine Synthase]

For the parameters CD163 and FKBP51, in addition, the time-dependent influence of CR ligands on the mRNA expression in human whole-blood cultures was examined. While prednisolone and the agonist in the transactivation, compound 1, induced the expression of these genes permanently, the antagonist in the transactivation, compound 2, did not lead to the increase of the gene expression.

[image]

[Key: Verbindung=Compound]

In addition, it was examined whether the antagonist in the transactivation can selectively prevent the gene induction by the standard glucocorticoid prednisolone for selected parameters for the transactivation activity.

Both prednisolone and compound 2 lead to the suppression of the mRNA expression of the transrepression parameters TNF-α and IL-1β in human PBMC cultures. The combination of the two GR ligands leads to results similar to the single dose.

[image]

In contrast to this, the transactivation parameters CD163 and FKBP51 are induced only by prednisolone, not, however, by the antagonists in the transactivation, compound 2. The simultaneous administration of compound 2 and prednisolone results in an induction of the transactivation parameters that is clearly lower in comparison to the prednisolone administered alone. Actually, the CD163 expression is reduced by compound 2 in all batches.

These data confirm that the selected parameters in the new test system that we defined, i.e., in unstimulated primary immune cells, are suitable for the detection of an antagonistic activity of selective GR ligands.

5.2 In Vivo Results in Mice

The reduction of the weight of the adrenal glands after an administration of the SEGRA substances over 5 days confirms that both substances are active in vivo in the selected dosage (30 mg/kg).

[image]

The in vivo regulation of the expression of selected genes was characterized in the spleen cells of SEGRA-treated mice after 24 hours.

The inhibition of the inflammatory cytokines as parameters for the transrepression was less pronounced for the antagonists in the transactivation, compound 2, than for the agonists, compound 1. The expression of these genes, however, was significantly inhibited by the two substances in comparison to the vehicle control.

[image]

Below, the results for the in vivo regulation of selected genes, whose induction correlates with the transactivation activity of GR ligands, are shown.

[image]

[Key: Glutamin synthetase=Glutamine Synthetase]

The treatment with the agonist in the transactivation, compound 1, results in a clear induction of the expression of FKBP51, CD163, GILZ and glutamine synthetase in the spleen cells. In contrast to this, the antagonist reduces the expression of these genes in the transactivation, compound 2.

For all selected parameters, not only a reduced induction but even an inhibition of the gene expression can be detected for the transactivation antagonists. It is probable that this effect is induced by the antagonism of the transactivation activity of the endogenic glucocorticoids.

In summary, based on two selected, selective GR agonists (SEGRA) of the Schering AG, it was possible to show that the study of the expression of defined genes and/or proteins in the test system of the unstimulated primary immune cells is suitable for characterizing the molecular mechanism of selective GR ligands with respect to their agonistic or antagonistic in vitro and in vivo activity in the transactivation or transrepression.

5.3 Evaluation of the Ratio According to Formula 2

The purpose was to characterize the extent of the dissociation of a substance by a value that reflects the varying influence of the expression of different transrepression and transactivation parameters in a summary.

To characterize the dissociated action of the SEGRA test substances, a dissociation factor was therefore defined that shows the dissociation of transactivation and transrepression activity based on the induction or suppression of selected genes.

As parameters that could [make] reference to the mechanism of the transrepression, TNF-α and IL-1β were selected. CD163 and FKBP51 are used to detect the transactivation activity.

Below, a ratio between the transrepression and transactivation parameters that are normalized to prednisolone was derived according to formula 2:

$$Ratio = \frac{\sqrt[2]{TNF - α \times IL - 1 β}}{\sqrt[2]{CD 163 \times FKBP 51}}$$

The factors for the transactivation and transrepression activity were averaged geometrically and put into a ratio with one another. According to this formula, a reduced gene induction of CD163 and FKBP51, with a suppression of TNF-α and IL-1β that is comparable to prednisolone, results in an increase of the ratio to values >1. The higher the ratio, the more dissociated the GR ligand is in comparison to prednisolone (ratio=1). The results are depicted in the following diagram.

[image]

Figure: Ratios for Visualization of the In Vitro Dissociation of the GR Ligands in Comparison to Prednisolone in PBMC Cultures (n—3) after 1, 2, 4, 12 and 24 Hours.

[Key to Figure:]

Ratio Transrepression/Transaktivierung=Transrepression/Transactivation Ratio

Prednisolon=Prednisolone

Dexamethason=Dexamethasone

Partialagonist in Transaktivierung=Partial Agonist in Transactivation

Stunde=Hour

Stunden=Hours

The ratios were calculated after the normalization of the gene expression values to prednisolone from the quotient of the geometric means of the parameters for transrepression (TNF-α and IL-1β) and for transactivation (CD163 and FKBP51). Mean values±SD.

In summary, a ratio (dissociation factor), which characterizes the molecular mechanism of the SEGRA test substances that is dissociated to differing degrees in comparison to prednisolone, could be drawn up from the gene expression values in PBMC cultures. In particular for the TA antagonists, a good persistence of the ratio increase (beginning with the 2-hour value) can be observed over time. Based on this dissociation factor, the standard GK prednisolone and dexamethasone are largely comparable in their transrepression and transactivation properties at all times.

5.4 In Vivo Tests

Kinetic Gene Expression in Spleen Cells of Mice Treated with GR Ligands

As in the in vitro kinetics test, a dissociation factor was calculated to visualize the dissociated in vivo action of the SEGRA test substances. These factors included the gene expression values for the parameters for the transrepression and transactivation normalized to the expression after prednisolone treatment (mean values of the prednisolone group at the various times).

Parameters for the gene suppression were TNF-α and IL-1β, and parameters for the gene induction were CD163 and FKBP51. The dissociation factor was calculated from the quotient of the geometric mean values of the transrepression and transactivation parameters corresponding to the explanatory formula 2.

[image]

Figure: Ratios for Visualization of the In Vivo Dissociation of the GR Ligands in Comparison to Prednisolone in the Spleen Cells of BALB/c-mice (n=5) After 2, 6 and 24 Hours.

[Key to Figure:]

Ratio Transrepression/Transaktivierung=Transrepression/Transactivation Ratio

Prednisolon=Prednisolone

Partialagonist in Transaktivierung=Partial Agonist in Transactivation

Stunden=Hours

The ratios were calculated after the normalization of the gene expression values to prednisolone from the quotient of the geometric means of the parameters for transrepression (TNF-α and IL-1β) and for transactivation (CD163 and FKBP51).

The figure above graphically represents the kinetics of the ratios for the dissociation in comparison to prednisolone for the SEGRA test substances. For all SEGRA test substances, the maximum in vivo dissociation could be detected 24 hours after administration. The minimum dissociation was observed after 6 hours for all SEGRA test substances. For the TA agonists and TA partial agonists, the values were even significantly lower in comparison to prednisolone.

In summary, the selected parameters have also proven suitable in vivo to represent the transrepression and transactivation activity of the GR ligands and the dissociated action of the TA partial agonists and the TA antagonists. As a whole, however, the in vivo dissociation in the spleen cells of mice was less and not as consistent as in the in vitro tests on human PBMC. At least partially responsible in this respect is the lower activity of the SEGRA test substances in the in vivo suppression of the transrepression parameters TNF-α and IL-1β.

5.5 Visualization of the Use of the Parameters to Detect an Antagonistic Action

The previous results showed only the varying agonism of GR ligands, i.e., e.g., a reduced or deficient induction of CD163 and FKBP51 by the antagonists in the transactivation ZK 243185.

The parameters can also be used to produce the antagonism directly by the TA-mediated gene induction being prevented in primary human immune cells by a standard glucocorticoid such as prednisolone, while the TR-mediated inhibition, e.g., the TNFa expression, is not impaired. That is to say, a dissociation in the antagonism is called for.

[image]

1. Process for the characterization of the transactivation and transrepression activity of glucocorticoid receptor (GR) ligands by gene and/or protein expression analysis of GR-sensitive genes, characterized in that the following steps are performed:
a) Exposure of primary immune cells to a GR ligand,
b) Detection of the regulation of the expression of at least one GR-sensitive gene to determine the transrepression,
c) Detection of the regulation of the expression of at least one GR-sensitive gene to determine the transactivation,
d) Normalization of the values that are obtained by reference to a known glucocorticoid,
e) Derivation of the ratio according to formula 1
$$Ratio = \frac{\sqrt[n]{{\prod }_{t = 1}^{n} {TR}_{t}}}{\sqrt[m]{{\prod }_{r = 1}^{m} {TA}_{r}}} .$$

2. Process according to claim 1, wherein for the characterization of the transactivation, the expression of IFN-γR1, TNF-R1, IL-1R1, IL-2Rα, IL-13Ra, CXCR4, GITR, β2-adrenoreceptor, hemoxygenase 1, IL-2, MIF, annexin 1, or thrombospondin 1 is determined.

3. Process according to claim 1, wherein for the characterization of the transactivation activity, the induction of CD163, FBKP51, glutamine synthase or GILZ is determined.

4. Process according to claim 1, wherein for the characterization of the transrepression activity, the suppression of the proinflammatory cytokines HLA-DR, CD86, IL-β, IL-8, TFN-α or Rantes is determined.

5. Process for the characterization of the transactivation and transrepression activity of glucocorticoid receptor (GR) ligands by gene and/or protein expression analysis of GR-sensitive genes, wherein the following steps are performed:
a) Exposure of primary immune cells to a GR ligand,
b) Detection of the regulation of the expression of TNF-α and IL-1β,
c) Detection of the regulation of the expression of CD163 and FKBP51,
d) Normalization of the values that are obtained by reference to a known glucocorticoid,
e) Derivation of the ratio according to formula 2
$$Ratio = \frac{\sqrt[2]{TNF - α \times IL - 1 β}}{\sqrt[2]{CD 163 \times FKBP 51}}$$

6. Process according to at least one of claims 1 to 5, wherein the primary immune cells are unstimulated.

7. Process according to at least one of claims 1 to 6, wherein the protein detection is carried out on the cells or after secretion in liquids.

8. Process according to at least one of claims 1 to 7, wherein the primary immune cells from lymphatic organs, from bone marrow or from blood are examined.

9. Process according to at least one of claims 1 to 8, wherein the blood in the living organism has been removed.

10. Process according to at least one of claims 1 to 9, wherein the normalization of the transrepresson and the transactivation parameters to prednisolone is carried out.

11. Use of the processes of the preceding claims for detecting the competitive or non-competitive, agonistic, partial agonistic, partial antagonistic or antagonistic activity of a GR ligand.

12. Use of the processes according to claims 1-10 in in vitro and in vivo experiments and as biomarker assays.
